(19) United States
(12) Patent Application Publication
(10) Pub. No.: US 2025/0144425 A1
(43) Pub. Date: May 8, 2025

(54) NEUROREHABILITATION SYSTEM AND METHOD OF NEUROREHABILITATION COMPRISING NEURALLY CONTROLLED STIMULATION

(71) Applicant: VIBRAINT INC., Richmond Hill (CA)

(72) Inventors: Ilia Borishchev, Richmond Hill (CA); Igor LAVROV, Richmond Hill (CA)

(21) Appl. No.: 18/937,916

(22) Filed: Nov. 5, 2024

Related U.S. Application Data
(60) Provisional application No. 63/596,409, filed on Nov. 6, 2023.

(51) Int. Cl. A61N 1/36; A61N 1/05; A61N 1/20

(52) U.S. Cl. CPC A61N 1/36103; A61N 1/0551; A61N 1/20; A61N 1/36025; A61N 1/36031; A61N 1/36139; A61N 1/3615; A61N 1/36167

(57) ABSTRACT

The system of neurorehabilitation and the method of neurorehabilitation belong to the field of medical rehabilitation and neurology and can be used as a system and method of rehabilitation of patients suffering sensorimotor deficit and/or neuropathic pain. The achievement of the invention is restoration of sensorimotor functions and reduction of neuropathic pain in paralyzed limbs by rehabilitation exercising with providing a neural biofeedback combined with multisensory stimulation, both controlled by the patient's neural activity, aimed at re-establishing of lost neural connections.

### DESCRIPTION OF INVENTION

### Field of Invention

The invention is directed to the field of medicine, and more specifically to a system and method of neurorehabilitation in rehabilitation of patients suffering from motor and sensory deficits and/or neuropathic pain due to disorders of the central nervous system, such as acquired brain injuries (ABI), stroke and traumatic brain injury, spinal cord injury, MS, ALS, sport injuries, orthopedic problems, and the like.

### Background and State of the Art

Stroke is a sudden and devastating condition affecting nearly one in six people. Up to 85% of survivors suffer from limb paralysis, and only 5% of them can restore full functional use of limbs. The number of people living with spinal cord injury—the second largest cause of paralysis—is at least fifteen million, increasing annually by about five hundred thousand patients.

The clinical standard-of-care for motor recovery focuses on the physical limbs, without significant attention to the neural origin or the resulting alterations in brain network dynamics. Studies have shown developing treatment strategies to facilitate neuroplasticity—a process of neural restoration—can direct the brain on how to create new neural pathways that can work around brain damage and alleviate physical and mental deficits. Recovery through facilitating neuroplasticity is especially effective during the first few weeks after disease, especially stroke, onset. However, other than standard medication and physiotherapy, there are almost no therapies that can effectively stimulate neural plasticity in patients with total or severe arm paralysis. Rehabilitation robots without neural control are often not effective enough due to lack of efficient neurofeedback and active rehabilitation components. The rehabilitation robots using neural control based on reading the electrical activity of paralyzed muscles are not effective enough in severe motor deficit.

This clear lack of early and efficient treatment options forms a gap in patient care worldwide. Besides immobility, paralyzed patients often experience social deprivation and depression due to their inability to live an independent life, while their caregivers are limited in their work, leisure, and travel time. When neuropathic pain onsets in addition to paralysis, it also rarely can be effectively treated thus increasing the patient's suffering and affecting their and their caregiver's physical and mental well-being.

Among the approaches to motor restoration (and often, reducing neuropathic pain) that strive to facilitate brain plasticity mechanisms, four technologies have received particular attention in recent years: brain-computer interfaces (BCI), virtual reality (VR), robotics and electrical stimulation. While VR paradigms allow manipulating visual information related to motor targets and body representation, BCIs provide direct access to the neural signals involved in motor control. A BCI samples activity in a brain area of interest and converts this activity into commands to an external device, for example a robotic orthosis for motor rehabilitation. Another method of neural control is electromyography control, when weaker than normal electrical activity of muscles is used to control impact to the body, stimulation, changes in the visual scene etc. As such, BCI, EMG, VR and robotic technologies could be used for motor rehabilitation as tools that enable motor behaviors that otherwise would be impossible in patients with deteriorated voluntary movements. To this end, such an approach is utilized where patients modulate their so-called sensorimotor mu rhythm by imagining movements, the BCI then translates these cortical modulations into limb motions augmented by an attached robotic device. Such motor-imagery BCIs are currently a mainstream rehabilitation approach that has proved to be effective, even though decoding motor imagery from EEG is a challenging problem.

To overcome this challenge, in our prior invention (U.S. application Ser. No. 17/439,800) we leveraged the so-called process of visual-motor transformation by using phenomena of so-called evoked cortical visual potentials for BCI control of a rehabilitation robotic device, which are also translated into movements of a virtual avatar. In the mentioned prior invention, we also claimed an electrical stimulator as a system component, and, accordingly, an electrical stimulation of the muscles and nerves responsible for moving the paralyzed limb accompanying the robotic impact on the trained limb.

In the current claim we further elaborate and introduce components, including invasive, and methods for electrical stimulation of motor neural tracts and muscles, including spinal cord and peripheral nerves (primary radial and medial, but not only), and brain structures, and introduce haptic (tactile) stimulator and haptic (tactile) stimulation of patient's body parts, both controlled by the patient's neural activity registered at the central and/or peripheral nervous system. We also introduce bimanual training aimed at facilitation of mastering neural control and on transfer of motor skills from healthy to affected limbs.

The proposed improvements in state-of-the-art are aimed at more intense activation of neural circuitries and pathways relevant to motor tasks, including, but not only, in patients with acute, subacute and chronic SCI and stroke and those suffering from severe damages of ascending and descending neural pathways, leading to restoration of affected sensorimotor functions.

### BRIEF DESCRIPTION OF INVENTION

In one aspect, the present invention provides a neurorehabilitation system for motor rehabilitation (restoration of movement) in patients whose mobility is affected by diseases and traumas of the central nervous system such as stroke, traumatic brain injury (TBI), spinal cord injury (SCI) and similar causing sensorimotor deficits in the limbs, primarily manifesting in immobility, loss of autonomic functions, and neuropathic pain. The system is provided with a plurality of visual display devices, a plurality of registering devices configured to register neural activities, and a plurality of stimulation devices for an electrical and tactile stimulation. In addition, the system includes a processor with a database and a software. The software is configured to record, recognize, extract, and interpret registered signals of the neural activities. Further, the software is configured to transmit commands formed on the basis of an interpretation of the registered signals of the neural activities to the at least one visual display device and/or to the at least one stimulation device, implementing feedforward control based on an anticipated change to modify visualization and/or stimulation to counteract the anticipated change.

In another aspect, the present invention provides a method of neurorehabilitation in patients whose mobility is affected by diseases and traumas of the central nervous system such as stroke, traumatic brain injury (TBI), spinal cord injury (SCI) and similar causing sensorimotor deficits in the limbs, primarily manifesting in immobility, loss of autonomic functions, and neuropathic pain. The method provides for presenting via a visual display device a task to be performed by trained objects, registering signals of neural activities by at least one registering device, and transmitting registered signals of neural activity to a processor with a software having or connected to a database. Further, the method can include recording, recognizing, extracting, and interpreting the registered signals necessary for interpretation of the neural activities by the processor, learning by the software to extract the registered signals necessary for interpretation of neural activity and forming the database, and interpreting the registered signals using the database. The method then provides for transmitting commands formed on the basis of the interpretation of the registered signals of the neutral activities to the visual display device and/or the stimulation devices and implementing feedforward control based on an anticipated change to modify visualization and/or stimulation to counteract the anticipated change and stimulating via an electrical stimulation the neural system of a patient by at least one stimulation device using at least one stimulation channel. Lastly, the method can include transmitting signals to the visual display device and presenting on the visual display device the task execution progress.

### BRIEF DESCRIPTION OF DRAWINGS

FIG. 1 shows a block diagram of a neurorehabilitation system according to embodiments of the present invention.

FIG. 2 shows a block diagram of a method of neurorehabilitation according to embodiments of the present invention.

FIG. 3 shows a view of the neurorehabilitation system and its implementation according to embodiments of the present invention.

FIG. 4 shows another view of the neurorehabilitation system according to embodiments of the present invention.

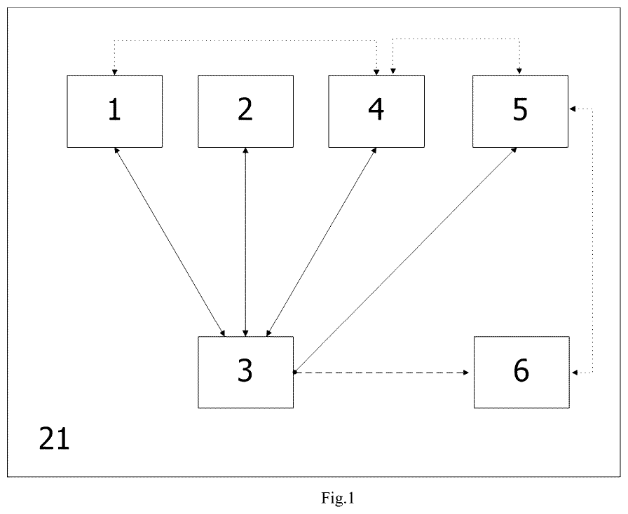

Below is the list of references for each feature shown in FIGS. 1-4.

Position 1—visual display device (—s);

Position 2—registering device (—s) for registering neural activity;

Position 3—computer with software and database;

Position 4—impact device (—s);

Position 5—electrical stimulator (—s);

Position 6—tactile stimulator (—s);

Position 7—preliminary (pre-exercising) electrical stimulation level

Position 8—permanent electrical stimulation during the exercise;

Position 9—visual presentation of the assigned task by the visual display device;

Position 10—registration of signals of neural activity;

Position 11—transmission of signals of neural activity to a computer with software and database;

Position 12—extraction and recognition of registered signals of neural activity by a computer and interpretation by comparison with a database;

Position 13—transmission of commands, based on the interpretation of the registered signals of neural activity, to an impact device for impacting the trained object;

Position 14—the impact of the impact device on the trained object in accordance with the recognized signals of neural activity;

Position 15—transmission of commands, based on the interpretation of the registered signals of neural activity, to an electrical stimulator to change stimulation parameters;

Position 16—electrical stimulation during the robotic-driven or voluntary motion of the limb;

Position 17—optional transmission of commands, based on the interpretation of the registered signals of neural activity, to a tactile stimulator to apply tactile stimulation;

Position 18—optional tactile stimulation

Position 19—transmission of the digital signal to the visual display device (—s);

Position 20—visual presentation of the execution of the task by the visual display device (—s);

Position 21—a neurorehabilitation system; and

Position 22—a method for neurorehabilitation.

### DETAILED DESCRIPTION OF INVENTION

Reference to “a specific embodiment” or a similar expression in the specification means that specific features, structures, or characteristics described in the specific embodiments are included in at least one specific embodiment of the present invention. Hence, the wording “in a specific embodiment” or a similar expression in this specification does not necessarily refer to the same specific embodiment.

Hereinafter, various embodiments of the present invention will be described in more detail with reference to the accompanying drawings. Nevertheless, it should be understood that the present invention could be modified by those skilled in art in accordance with the following description to achieve the excellent results of the present invention. Therefore, the following description shall be considered as a pervasive and explanatory description related to the present invention for those skilled in the art, not intended to limit the claims of the present invention.

Reference to “an embodiment,” “a certain embodiment” or a similar expression in the specification means that related features, structures, or characteristics described in the embodiment are included in at least one embodiment of the present invention. Hence, the wording “in an embodiment,” “in a certain embodiment” or a similar expression in this specification does not necessarily refer to the same specific embodiment

The object of the present invention is the system and method of motor rehabilitation (restoration of movement) in patients whose mobility is affected by diseases and traumas of the central nervous system such as stroke, traumatic brain injury (TBI), spinal cord injury (SCI) and similar causing sensorimotor deficits in the limbs, primarily manifesting in immobility, loss of autonomic functions, and neuropathic pain.

The present invention supports and promotes a restoration of neural control and mobility in paralyzed limbs by forming neurofeedback connections between the patient's intention to move the limb and its execution and by excitation of neural pathways during active rehabilitation exercising with neural control.

The neurorehabilitation system of this invention can include:
a visual display device (—s), a device (—s) for registering neural activity, a stimulation device (—s) that can be electrical and/or tactile (haptic), an optional device (—s) for impacting a paralyzed limb that can also be further referred to as a trained object (—s) or trained limb (—s), a computer, a database, a software for recognizing, extracting and recording of the registered signals of neural activity and interpreting them using the database. The computer with the software is capable of transmitting commands formed on the basis of the interpretation of the registered signals of neural activity to the visual display device, to the stimulation device (—s) and to the impact device (—s).

According to embodiments of the present invention, the visual display device can be a virtual reality headset. An electroencephalograph (EEG) can be used as a device for registering neural activity. An impacting device can be a robotic device moving a trained limb. A transcutaneous electrical stimulator of spinal cord and muscles and a vibrational soft plate can be used as electrical and tactile stimulation devices respectively.

According to embodiments of the present invention, the neurorehabilitation method using embodiments of the neurorehabilitation system for rehabilitation of, for example, upper limbs can include the following steps:
measuring of the excitation threshold of the neuromuscular system and neural tracts and circuits by sending series of electrical impulses with changing parameters such as amplitudes, frequencies, shapes, combination of engaged channels etc., towards the spinal cord by means of electrical stimulator, till the muscular or neural response suggests that the sensorimotor system, neural tract or its part is excitated;
preliminary prolonged excitation (“warming up”) of the sensorimotor system and neural circuits by permanent delivery of electrical stimulation to the area of the spinal cord or peripheral nerves and muscles, so the neural circuits, facilitated directly or by afferents that innervates the trained limb (a paralyzed arm), send the signals to the corresponding muscles;
permanent delivery of electrical stimulation to the area of the spinal cord where the neural tract innervating the trained limb (a paralyzed limb) leaves the spinal cord, and/or of the muscles responsible for moving the trained limb, with the amplitude below the excitation threshold (subthreshold level), during the whole rehabilitation exercise except periods when the trained limb is being driven by a robotic device;
presentation on the virtual reality headset of the task (—s) requiring movement of the trained limb (such as pointing the arm towards an object, grasping it etc.);
registration of signals of neural activity;
transmission of registered neural activity signals to the computer with software associated with the database;
extracting signals of neural activity necessary for the interpretation by the computer with software;
interpretation of the selected signals by comparison with to database in order to detect signal patterns related to desired motor activity;
transmission of the command formed on the basis of the interpretation of recorded signals of neural activity to a robotic device for impacting the trained limb (—s);
the impact of the robotic device on the trained limb (—s) in accordance with the received command in order to move it into desired position, optionally alternating with voluntary motion of the corresponding intact (healthy) limb driven by patient's own muscular effort;
transmission of the commands formed on the basis of the interpretation of recorded signals of neural activity to stimulation devices;
electrical stimulation of the spinal cord area where the neural tract that innervates the trained object leaves the spinal cord, and/or the muscles responsible for moving the limb with the amplitude above the excitation threshold (suprathreshold level) during the robotic driven motion of the trained limb in accordance with the received commands or voluntary motion of the healthy limb;
tactile stimulation of the parts of the patient's body (such as lower neck or shoulders, or parts of the trained limb) having sufficient haptic sensitivity and corresponding the same side (right or left) as the moved limb, during the robotic driven motion of the trained limb in accordance with the received commands or during the voluntary motion of the healthy limb;
transmission of the signal to the virtual reality headset;
visual presentation to the patient of the task execution progress (e.g. the motion of the arm towards the object).

As a result, a biofeedback formed between the motor intention and its sensation during physical motions and observing motions of the virtual avatar via visual, proprioceptive, kinesthetic and haptic channels, optimizing the performance of the motor tasks and motor training, stimulating the neuroplasticity across brain, spinal cord, and afferent and efferent pathways, promoting restoration of neurologic function, sensorimotor integration, and reduction of neuropathic pain. It is also expected to reduce the level of adverse events such as motor sickness when using VR in rehabilitation

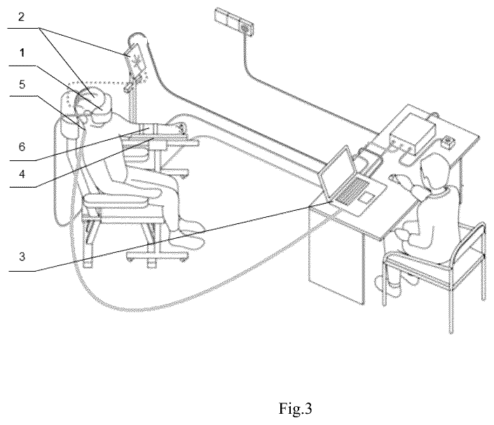

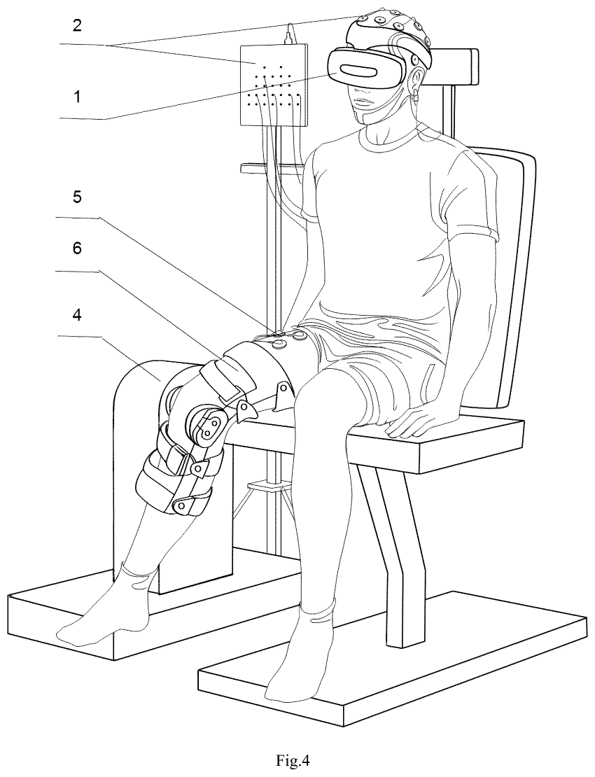

FIG. 1 illustrates a neurorehabilitation system 21 according to embodiments of the present invention. The neurorehabilitation system 21 can be provided with a visual display device 1, which can be a virtual reality headset (as shown in FIGS. 3 and 4). The visual display device 1 allows patient's immersion in a virtual environment by visually presenting the task (for example, movement of a limb), the process of performing the task by the patient and its completion to involve the patient in a virtual process that, along with the proprioceptive sense, makes the brain “believe” in voluntary connection between the intention to make a movement of the trained object (i.e. the paralyzed arm) and its physical movement, which is driven by a robotic device 4.

Establishment of such a connection contributes to more effective restoration of the motor function based on biofeedback and neuroplasticity mechanisms.

To facilitate active training, the system 21 can display a visual animated guidance of a task execution—a virtual phantom of the limb “performing” assigned movement via visual display 1. Additionally, the system 21 can display the progress of the task “from the third person view” thereby stimulating activation of mirror neurons, which facilitates restoration of neural connections. To motivate the patient, the display 1 can also show a number of successful attempts, play encouraging video and audio messages or the like.

The neurorehabilitation system 21 can be provided with a device(—s) for registering neural activity 2 (shown in FIG. 1), which registers and transmits signals of the brain and/or muscles activity to the computer 3, thereby forming a neural-computer interface to register, record, process, extract and interpret neural activities in order to determine the patient's cortical activity related to intention of moving the limb and generate a command to the impact (robotic) device (—s) 4 to execute the desired motion. An electroencephalograph (EEG) (as in example shown at FIG. 3 and FIG. 4), a near-infrared spectroscopy (NIRS), a magnetoencephalographic (MEG), a magnetic resonance imaging (MRI) or similar devices that read and register magnetic fields arising from the electrical activity of the brain can be used for registering brain activity. The system can include an electromyography (EMG) component, to measure electrical activity in muscles that set the trained object in motion corresponding to the level of their contraction with sensors placed on the skin or inside the muscles. Such neural signal registering devices can also be used in various combinations; a combination of several signal registering technologies can be also implemented in a single device.

The neurorehabilitation system 21 also can include a computer 3 with software for recording, extracting, recognizing and interpreting neural signals using a database. It then transmits the commands formed on the basis of the interpretation of the registered signals of neural activities to an impact device (—s) 4, stimulation devices 5 and 6, and to the visual display device (—s) 1 according to the transmit-receive principle thus forming a neural-machine interface.

The neurorehabilitation system 21 can be provided with a robotic device 4 (—s), which is used for physical interaction (impact) with a trained object, that is, a paralyzed, paretic, rehabilitated limb, including for moving the trained object in accordance with the recognized signals of neural activity. A variety of the implementation of the robotic device (—s) 4, including an exoskeleton, is possible. The robotic device can be used for training and rehabilitation of the upper (as shown in FIG. 3), lower (as shown in FIG. 4) limbs, as well as certain parts of the limbs such as fingers, palm, shoulder, foot and the like. In the following description an exoskeleton of the upper limb (as shown in FIG. 3) will be used as an example of a robotic device 4.

The system 21 can also include an electrical stimulator (—s) 5 to stimulate the motor neural tracts or/and muscles that set the trained object in a movement. The stimulation can be delivered with different signal types, shapes, and amplitudes at different channels (as outlined above), both during active exercising and before, via one or several stimulation electrodes, located at multiple sites (multisite stimulation) and oriented in different ways relative to the anatomical structures to achieve optimal levels of excitation on neural structures and pathways at each moment. The stimulation can be delivered to the surface of the skin, or inside the neural tracts and/or muscles and/or cortical structures, to excitate brain structures, spinal cord structures, and peripheral nerves respectively. The stimulation can be delivered with different timing patterns, such as simultaneously, or consecutive, or sequentially, alternating etc. It is possible that at different stimulation sites stimulation current can be alternating (AC) or direct (DC). The stimulation can also be delivered to cortical structures, invasively and/or non-invasively.

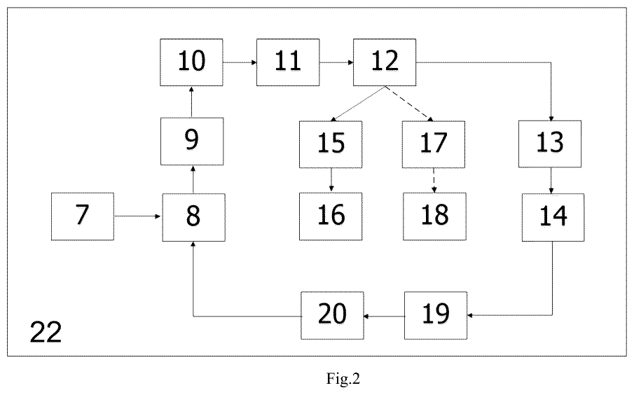

FIG. 2 illustrates a method of neurorehabilitation 22 using the system 21. As illustrated in FIG. 2, according to embodiments of the present invention, to “warm up” (to excitate) the neural tracts before the active exercising with neural control, a preliminary stimulation 7 at the suprathreshold or subthreshold level can be delivered for a prolonged period. After this preliminary suprathreshold stimulation, the active exercising with voluntary neural control begins. It is also possible that suprathreshold or subthreshold stimulation will be delivered for a prolonged period after exercising.

During the active movement of the trained object (e.g. a limb) with involvement of voluntary neural control, in order to enhance and enforce biofeedback on motor intention, during the movement of the trained object driven by the robotic device (—s) 4, the permanent simultaneous electrical stimulation 8 is delivered with the suprathreshold current amplitude. On the other hand, when the trained object is resting, the stimulation is being delivered with the subthreshold current amplitude. Such a stimulation delivery method with alternating amplitude further helps formation of patterns of neuronal activity corresponding to the implementation of desired movements of the trained object. The stimulation during the motion can also be delivered in a form of “wave” of suprathreshold amplitude propagating one-by-one along the sequence of electrodes, thus mimicking natural propagation on neural impulses.

Electrical stimulation (position 8) can be delivered to the spinal cord by a plurality of electrodes (not shown) located at different levels of the spinal cord (such as cervical, thoracis, lumbar). The electrodes' locations can be determined by anatomical coordinates, neurophysiological assessment, and response to the stimulation.

According to embodiments of the present invention, as illustrated in FIG. 2, the system 21 can also be provided with a tactile (haptic) stimulator 6 to stimulate the sensorimotor system when the trained object is in the movement, by applying tactile stimuli to the patient's skin at the side corresponding to the limb being moved and/or opposite (contralateral) limb, at the area with sufficient or intact haptic function (such as lower neck or shoulders or the like). Such stimulation is aimed to provide haptic confirmation of the motion in situations when the limb's or its part's proprioceptive system is not sufficient due to severe paralyzation. This stimulation also acts on ‘trigger points’ facilitating the motor control and functional recovery. The haptic stimulation can be delivered via one or several stimulation actuators (not shown). In particular and not limiting, the haptic stimulation can be delivered in the form of vibration or slight pressure, or both, with different signal forms and amplitudes at different channels, including, but not limited to a series of consecutively starting impacts from several actuators thereby forming “waves” moving along the stimulated zone, for example in a direction of movement in order to further affirm the sensation.

A position 9 shows a visual presentation of the assigned task by the visual display device 1. For example, the visual presentation can be the motion of the arm or another limb of the virtual avatar towards the virtual object.

In a position 10, the signals of the neural activities are registered by the registering device 2 and the registered signals are then transmitted (a position 11) to the computing device (computer 3). A position 12 illustrates extraction and recognition of registered signals of neural activities by the computer 3 and interpretation of the registered signals using a database.

A position 13 illustrates the transmission of commands, based on the interpretation of the registered signals of neural activities, to the robotic device 4 for impacting the trained object. A position 14 illustrates an impact of the robotic device 4 on the trained object in accordance with the recognized signals of neural activities.

A position 15 illustrates a transmission of commands, based on the interpretation of the registered signals of neural activities, to an electrical stimulator 5 to apply suprathreshold stimulation. A position 16 illustrates the electrical stimulation at the suprathreshold level during the robotic-driven or voluntary motion of the trained object.

Optionally, another transmission of commands (a position 17), based on the interpretation of the registered signal of neural activity, can be carried out to a tactile stimulator to apply tactile stimulation, as described above. A position 18 illustrates the tactile stimulation during the robotic-driven or voluntary motion of the trained object.

A position 19 illustrates a transmission of the digital signal to the visual display device 1. In a position 20, a visual presentation of the execution of the task by the visual display device 1.

### EXAMPLES

Below is an example to illustrate the implementation of the neurorehabilitation system 21 and the functionalities of its elements. This example is to provide an opportunity for a person skilled in the art to understand the principles of interaction of system elements and the principles of operation and functioning of the system 21 as a whole and should be considered as illustrative and not limiting the scope of the invention. The following description is based mainly on FIG. 1 showing a block diagram of the interaction of the elements of the neurorehabilitation system and FIG. 2 showing the work diagram (a sequence of actions).

An operator (e.g., a medical practitioner) measures the excitation threshold of the neural circuits corresponding to the trained limb, and then sets electrical stimulation at the suprathreshold level (about 110-150% of the threshold value) for the period between 5 and 30 minutes to excitate (“warm up”) the sensorimotor neural tracts. This makes the sensorimotor neural tracts more responsive during following shorter periods of suprathreshold stimulation. The shorter periods can be about 3-10 seconds and accompany the motion of the limb (i.e., the trained object). If this is a multisite stimulation, the amplitude and current patterns can vary among sites.

During the active training of the limb, electrical stimulation at the subthreshold level (at about 50-90% of the threshold value) is being permanently delivered to the patient's neural circuits, tracts, and\or muscles, except periods when the limb is in motion, either robotic or voluntary driven.

Using the visual display device 1, the system assigns the patient with tasks consisting of action with objects in virtual reality requiring movements of the trained limb. In the course of performing the task, the patient has to imagine the movement of the limb to the selected target, to the selected position or in the selected direction, or interaction with the object (e.g. grasping), or focusing the attention on the target object or similar task. At the same time, the registering device (—s) 2 (e.g. EEG) is registering the signals of neural activity by analyzing which a software classifier at the computer 3 identifies patterns related to the desired movement or its target.

If the patient's own muscle activity in a paralyzed limb is strong enough (or has restored enough), the level of self-effort made by the patient's muscles can be set as a second or single condition for starting the robotic-driven movement. An electromyograph (as a component of the signal recording device 2) with sensors placed on the muscles that set the limb in motion measures their tension while the patient is trying to perform a desired movement. When the tension reaches the set threshold, the robotic motion begins.

Thereafter, a command is issued to the robotic device (—s) 4 (for example, an exoskeleton) to impact the limb—for example, to move the arm towards the target object and then grasp it in accordance with the detected intention of the patient. Simultaneously, the software on the computer 3 sends commands to stimulation devices 5 and 6 to increase electrical stimulation level to suprathreshold for the period of time when the robotic device 4 is in motion, and to apply haptic stimulation to a patient's skin in a sensitive area for the same period.

At the same time, the patient observes on a visual display device 1 (e.g., virtual headset) the motion of the virtual avatar executing the task of the exercise, corresponding and synchronous to the motion of the physical limb driven by the robotic device 4 and to the stimulation applied to neural tracts or muscles.

By using the elements and methods described above, the claimed invention supports restoration of motor control and reduction of neuropathic pain in paralyzed limbs by formation of neural biological feedback on the patient's intention to make a movement by its implementation and confirmation via multiple sensory inputs, resulting in improvement of neuroplasticity—a process in the brain that forms bypass neural pathways to replace those lost or damaged as a result of the disease.

Furthermore, by incorporating novel artificial intelligence (AI) and machine learning (ML) tools into rehabilitation and neurostimulation protocols described above to continuously analyze key parameters of exercising and patient performance, and physiological responses to more precisely predict patterns and their changes over time, will allow real-time adjustments in neurostimulation and impact parameters. This dynamic adaptation of the stimulation parameters and training protocols will further optimize recovery, improve neuroplasticity, and enhance therapeutic outcomes by personalizing the interventions based on the individual's current physical state, progress and needs.

The foregoing detailed description of the embodiments is used to further clearly describe the features and spirit of the present invention. The foregoing description for each embodiment is not intended to limit the scope of the present invention. All kinds of modifications made to the foregoing embodiments and equivalent arrangements should fall within the protected scope of the present invention. Hence, the scope of the present invention should be explained most widely according to the claims described thereafter in connection with the detailed description, and should cover all the possibly equivalent variations and equivalent arrangements.

The present invention can be a system, a method, and/or a computer program product. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a hard disk, a flash memory, an optical memory, a quantum memory, a random-access memory (RAM), a static random access memory (SRAM), and any suitable combination of the foregoing or the like. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network, or in a cloud storage. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an programming languages such as Java, JS, C++, C#, Python or the like, and other conventional procedural programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the present invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form described. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What we claim is:

1. A neurorehabilitation system comprising:
a plurality of visual display devices;
a plurality of registering devices configured to register neural activities;
a plurality of stimulation devices for an electrical stimulation; and
a processor with a database and a software configured to record, recognize, extract, and interpret registered signals of the neural activities,
wherein the software is configured to transmit commands formed on the basis of an interpretation of the registered signals of the neural activities to the at least one visual display device and/or to the at least one stimulation device, implementing feedforward control based on an anticipated change to modify visualization and/or stimulation to counteract the anticipated change.

2. The neurorehabilitation system according to claim 1 further comprises a plurality of impacting devices configured to impact trained objects,
wherein the software is configured to transmit commands formed on the basis of the interpretation of the registered signals of the neural activities to the impacting devices implementing feedforward control based on the anticipated change to modify impact to counteract the anticipated change.

3. The neurorehabilitation system according to claim 1, wherein at least two different registering devices are used simultaneously.

4. The neurorehabilitation system according to claim 1, wherein at least one of the registering devices is configured to register a brain neural activity.

5. The neurorehabilitation system according to claim 1, wherein at least one of the registering devices is configured to register a muscular neural activity.

6. The neurorehabilitation system according to claim 1, wherein at least two different stimulation devices are used simultaneously.

7. The neurorehabilitation system according to claim 1, wherein at least one of the devices for registering the neural activities comprises at least one invasive element.

8. The neurorehabilitation system according to claim 1, wherein at least one of the stimulation devices is a spinal cord electrical stimulation device which has at least one stimulation channel.

9. The neurorehabilitation system according to claim 1, wherein at least one of the stimulation devices is a functional electrical stimulation device which has at least one stimulation channel.

10. The neurorehabilitation system according to claim 1, wherein at least one of the stimulation devices is a tactile (haptic) stimulation device which has at least one stimulation channel.

11. The neurorehabilitation system according to claim 1, wherein at least one of the stimulation devices comprises more than one stimulation channel.

12. The neurorehabilitation system according to claim 1, wherein at least one of the stimulation devices comprises at least one invasive stimulation element.

13. The neurorehabilitation system according to claim 1, wherein at least one stimulation device is configured to use the registered signals by at least one registering device for configuring stimulation parameters.

14. The neurorehabilitation system according to claim 12, wherein the at least one stimulation parameter of at least one stimulation device can be configured based on the registered signals by at least one of the registering devices.

15. The neurorehabilitation system according to claim 2, wherein at least one impacting device can use the registered signals by at least one registering device for configuring impact parameters.

16. The neurorehabilitation system according to claim 15, wherein the at least one impact parameter of at least one of the impacting devices can be configured based on the registered signals by at least one of the registering devices.

17. The neurorehabilitation system according to claim 1, wherein the plurality of visual display devices comprises a single visual display device and/or the plurality of registering devices comprises a single registering device and/or the plurality of stimulation devices comprises a single stimulation device.

18. A method of neurorehabilitation, the method comprising:
presenting via a visual display device a task to be performed by trained objects;
registering signals of neural activities by at least one registering device;
transmitting registered signals of neural activity to a processor with a software having or connected to a database;
recording, recognizing, extracting, and interpretation the registered signals necessary for interpretation of the neural activities by the processor;
learning by the software to extract the registered signals necessary for interpretation of neural activity and forming the database;
interpreting the registered signals using the database;
transmitting commands formed on the basis of the interpretation of the registered signals of the neutral activities to the visual display device and/or the stimulation devices and implementing feedforward control based on an anticipated change to modify visualization and/or stimulation to counteract the anticipated change;
stimulating via an electrical stimulation the neural system of a patient by at least one stimulation device using at least one stimulation channel;
transmitting signals to the visual display device; and
presenting on the visual display device the task execution progress.

19. The method of neurorehabilitation according to claim 18 further comprises impacting at least one trained object by at least one impact device and implementing the feedforward control based on the anticipated change to modify impact to counteract the anticipated change.

20. The method of neurorehabilitation according to claim 18, wherein at the step of extraction and recognition of the registered signals of the neural activities of at least one of the signals of neural activity is selected from the group consisting of: a cortical visual evoked potential, a cortical motor imagination, a neuromuscular activity, evoked responses from the spinal cord and/or brain, electrically evoked compound action potentials, or mechanical and kinematical correlates of motor activity.

21. The method of neurorehabilitation according to claim 19, wherein the results of extraction and recognition of the registered signals of the neural activities are used to control at least one impact device or/and at least one stimulation device comprising at least one channel each.

22. The method of neurorehabilitation according to claim 21, wherein the registered signals of the neural activities are used to control the at least one impact device and/or at least one stimulation channel of at least one stimulation device based on a stage of a visual-motor transformation process determined using the registered signals of the neural activities.

23. The method of neurorehabilitation according to claim 20, wherein at least one stimulation parameter on at least one stimulation channel of at least one stimulation device is being changed based on the stage of a rehabilitation training exercise execution and/or on a stage of visual-motor transformation process determined using the registered signals of the neural activities.

24. The method of neurorehabilitation according to claim 20, wherein the registered signals of the neural activities are used to control stimulation channels of at least one stimulation device in a way mimicking the natural propagation of the neural signals during a voluntary muscular action based on the stage of the visual-motor transformation process determined using the extracted signals of neural activity.

25. The method of neurorehabilitation according to claim 19, wherein a stimulation by at least one channel of at least one stimulation device is synchronized with an action of at least one impact device and/or with visual presentation on a visual display device of the task being performed.

26. The method of neurorehabilitation according to claim 18, wherein the excitability threshold of the sensorimotor system of a patient or its part towards which the stimulation during the exercise is directed is being measured before rehabilitation by sending consecutive stimuli with increasing amplitude and measuring responses of the sensorimotor system after every stimulus to determine that the sensorimotor system reached the excitation threshold.

27. The method of neurorehabilitation according to claim 18, wherein before the start of the rehabilitation, a preliminary stimulation by at least one channel of at least one stimulation device is delivered with a stimulation amplitude above the excitation threshold of the sensorimotor system or its part towards which stimulation is primarily directed, in order to excitate the later before exercising.

28. The method of neurorehabilitation according to claim 19, wherein the stimulation by at least one channel of at least one stimulation device is synchronized with action of at least one impact device such that when the impacting action is ongoing, the stimulation amplitude is set above the excitation threshold of the sensorimotor system or its part towards which stimulation is directed, and when there is no ongoing impacting action, the stimulation amplitude is set below the excitation threshold.

29. The method of neurorehabilitation according to claim 18, wherein the stimulation is being consecutively delivered by at least two channels of at least one stimulation device with changing amplitudes, so that at the same time at some channels the amplitude is below and at some channels above the excitation threshold of the sensory system or its part towards which stimulation is primarily directed, thereby forming a wave propagating down the neural pathways.

30. The method of neurorehabilitation according to claim 18, wherein the patient is alternately given motor tasks to execute either by an affected limb driven by the impact device or by the corresponding intact limb driven by the own muscular effort

31. The method of neurorehabilitation according to claim 18, further comprising applying the electrical stimulation by at least two channels below and above the level of the spinal cord injury lesion.

32. The method of neurorehabilitation according to claim 31, wherein the electrical stimulation by at least two channels is applied at a cervical spinal cord level, a thoracic spinal cord level, or/and a lumbar spinal cord level.

33. The method of neurorehabilitation according to claim 31, wherein the electrical stimulation by at least one channel is applied at a spinal cord injury sublesional level.

34. The method of neurorehabilitation according to claim 18, wherein the electrical stimulation by at least two channels is applied to peripheral nerves and to the spinal cord respectively.

35. The method of neurorehabilitation according to claim 18, wherein the electrical stimulation by at least two channels is applied to the spinal cord and to the brain cortex respectively.

36. The method of neurorehabilitation according to claim 18, wherein the electrical stimulation by at least two channels is applied to the spinal cord by electrodes placed in accordance with orientation of the spinal cord neural roots to achieve the optimal level of neural excitation.

37. The method of neurorehabilitation according to claim 31, wherein a timing of the electrical stimulation by at least two stimulation channels placed at the multiple spinal cord levels is determined to achieve the optimal level of neural excitation at each level at each moment.

38. The method of neurorehabilitation according to claim 37, wherein the electrical stimulation by at least two stimulation channels placed at multiple spinal cord levels can be delivered simultaneously, or consecutive, or sequentially, or alternating.

39. The method of neurorehabilitation according to claim 18, wherein the electrical stimulation by at least two channels is delivered in a form of alternating current stimulation by at least one channel, and direct current stimulation by at least one channel.

40. The method of neurorehabilitation according to claim 18, wherein the electrical stimulation by at least one channel is delivered with a subthreshold current amplitude for a prolonged period of time, prior to or after the rehabilitation in accordance with claim 18.

41. The method of neurorehabilitation according to claim 40, wherein the electrical stimulation by at least one channel is increased to a suprathreshold current amplitude level after a period of stimulation at the subthreshold current amplitude level.
